(19) United States
(12) Patent Application Publication
(10) Pub. No.: US 2006/0178348 A1
(43) Pub. Date: Aug. 10, 2006

(54) Compositions and therapeutic methods utilizing a combination of a protein extravasation inhibitor and an NSAID

(75) Inventor: John R. Plachetka, Chapel Hill, NC (US)

Correspondence Address: FITCH, EVEN, TABIN & FLANNERY, P. O. BOX 65973, WASHINGTON, DC, 20035, US

(73) Assignee: POZEN Inc., Chapel Hill, NC (US)

(21) Appl. No.: 11/331,036

(22) Filed: Jan. 13, 2006

Related U.S. Application Data
(60) Provisional application No. 60/645,598, filed on Jan. 24, 2005.

(51) Int. Cl. A61K 31/60; A61K 31/415; A61K 31/405; A61K 31/365; A61K 31/192

(52) U.S. Cl. 514/165; 514/406; 514/471; 514/563; 514/570; 514/569; 514/420

(57) ABSTRACT

The present invention is directed to compositions containing an anti-inflammatory compound that acts by blocking protein extravasation together with an NSAID. These compositions may be used to treat inflammation and pain, especially headache pain. The invention also includes methods in which these drugs are separately administered to a patient.

### CROSS REFERENCE TO RELATED APPLICATIONS

The present application claims priority to, and the benefit of, U.S. provisional application 60/645,598, filed on Jan. 24, 2005. The contents of this prior application are hereby incorporated by reference in its entirety.

### FIELD OF THE INVENTION

The present invention is directed to pharmaceutical compositions that contain an agent that acts by preventing protein extravasation together with a nonsteroidal anti-inflammatory drug (NSAID). The pharmaceutical compositions may be used in treating pain and inflammation and will be particularly useful in the treatment of headache.

### BACKGROUND OF THE INVENTION

Substance P is an endogenous 11 amino acid peptide that acts as a neurotransmitter and neuromodulator in the central and peripheral nervous systems. It is believed to participate in nociceptive pathways by slowing the depolarization of sympathetic ganglia, and to act as a co-transmitter for enteric neurons. Substance P also acts on receptors in the lung to increase vascular permeability and induce mucus secretion. In addition, it appears to be involved in inflammatory conditions, such as arthritis, asthma, hay fever, inflammatory bowel disease and migraine.

Various peptide analogs of Substance P with receptor antagonist properties have been described and used in experimental studies. In 1991, a non-peptide tachykinin antagonist (CP96345) was developed from a lead obtained by random screening and there are now several tachykinin antagonists that distinguish between receptor subtypes. The anti-inflammatory action of NK-1 antagonists is supported by studies showing that these antagonists block neuropeptide release and dural plasma protein extravasation (Buzzi, et al., Cephalalgia 15:277-280 (1995); Buzzi, et al., Pathol. Biol. 40:313-317 (1990)). These findings have led some scientists to conclude that NK-1 antagonists that are active in models of cranial inflammation but which do not induce vasoconstriction might relieve migraine pain without the side effects characteristic of triptans. However, clinical studies using drugs specific for neurogenic inflammation have, in general, produced poor results in terms of efficacy (Goldstein, et al., Lancet 358:1230-1234 (2001); Ramadan, Curr. Med. Res. Opin. 17(suppl. 1):S75-S80 (2001); Roon, et al., Ann. Neurol. 47:238-241 (2000); Goldstein, et al., Clin. Pharmacol. Ther. 67:419-426 (2000); Goldstein, et al., Cephalalgia 17:785-798 (1997); Goldstein, et al., Cephalalgia 21:102-106 (2001); and May, et al., Expert Opin. Investig. Drugs 10:673-678 (2001)).

A different group of drugs that has been used to treat inflammation and pain, including migraine pain, is the nonsteroidal anti-inflammatory drugs (NSAIDs). Unlike NK-1 antagonists, NSAIDs act primarily by blocking the activity of cyclooxygenase (COX) enzymes, thereby inhibiting the production of pro-inflammatory prostaglandins. In general, NSAIDs are advantageous for many conditions because they have proven to be effective in alleviating existing symptoms of inflammation and pain, rather than preventing the development of new symptoms.

### SUMMARY OF THE INVENTION

The present invention is based upon the concept that NSAIDs and agents that reduce inflammation by inhibiting extravasation will have a complementary effect in relieving pain, particularly pain that accompanies inflammation. The NSAIDs block the action of pro-inflammatory agents that have already leaked from blood vessels, whereas agents reducing extravasation block the further release of pro-inflammatory substances. Thus, the NSAIDs can provide for the rapid relief of inflammatory pain and both the NSAIDs and extravasation inhibitors act together to provide longer term relief from inflammation and pain.

In its first aspect, the invention is directed to a pharmaceutical composition in unit dose form that contains an anti-inflammatory compound that is not an NSAID and that acts by blocking protein extravasation (e.g., as determined using the assay of Markowitz, et al. (J. Neurosci. 7:4129-4136 (1987)) together with an NSAID. Each of these drugs should be present in an amount effective to relieve pain upon the administration of one or more of the unit doses to a patient, and a synergistic effect should generally be evident at doses of the extravasation blocker that appear to provide little or no clinical benefit when used alone.

Preferred compounds blocking protein extravasation are neurokinin-1 (NK-1) antagonists. Antagonists suitable for use in the present invention have been described in numerous references. Many of these references along with specific compounds are set forth below in the Detailed Description of the Invention section.

NSAIDs that can be used include: aspirin; ibuprofen; acetaminophen; naproxen; diclofenac; ketorolac; etodolac; flurbiprofen; oxaprozin; indomethacin; mefenamic acid; nabumetone; piroxicam; celecoxib; rofecoxib; valdecoxib; and lomoxicam. These should, in general, be present in an amount of between 1 and 600 mg, whereas the extravasation blocker should generally be present in an amount of between 0.5 and 600 mg, and preferably at between 10 and 400 mg. These drugs may also be present in an amount sufficient to provide for therapeutic synergy or migraine-related therapeutic synergy, as these terms are defined herein. The most preferred NSAIDs for use in pharmaceutical compositions are: aspirin; ibuprofen; acetaminophen; naproxen; diclofenac; ketorolac; lomoxicam; and etodolac. Unless otherwise indicated, it is intended that any pharmaceutically acceptable form or salt of a drug referred to herein may be used in compositions and methods, and that the weights provided refer to the free form of the drug.

In another aspect, the present invention is directed to methods for treating a patient for pain or inflammation by administering a therapeutically effective amount of any of the pharmaceutical compositions described above. The pain or inflammation may be the result of arthritis, medical or dental procedures, cancer, injury, infection, etc. Preferably, the compositions will be used to treat patients for migraine.

The invention also includes treatment methods in which a patient is administered a therapeutically effective amount of an NSAID and separately administered a therapeutically effective amount of an anti-inflammatory compound that acts by blocking protein extravasation, preferably an NK-1 antagonist. The NSAID and anti-inflammatory compound should be administered in a co-timely manner, i.e., they should be administered in close enough temporal proximity that their therapeutic effects overlap. Preferably, the administration of these compounds occurs within two hours of one another. The most preferred compounds and preferred dosages are the same as those discussed above in connection with pharmaceutical compositions.

In cases where patients are treated for migraine, the administration of NSAID may be preceded by a step in which a gastric prokinetic agent, preferably metoclopramide, is given to the patient at a dose of between 5 and 40 mg. In general, it is preferred that the metoclopramide be given between 10 and 30 minutes prior to NSAID, but the drugs may also be given concurrently if desired. Alternatively, metoclopramide may be included as part of unit dosage forms which may be, optionally, coordinated to release the metoclopramide first (see U.S. Pat. No. 6,479,551).

This procedure may be used to treat pain, inflammation, and headaches falling into a wide variety of classes including: migraine headache; tension-type headache; cluster headache and chronic paroxysmal hemicrania; miscellaneous headache unassociated with a structural lesion; headache associated with a non-vascular intracranial disorder; headache associated with the administration of a substance or its withdrawal; headache associated with noncephalic infection; headache associated with a metabolic disorder; headache associated with a disorder of the cranium, neck, eyes, ears, nose, sinuses, teeth, mouth or other facial or cranial structure; cranial neuralgias; and nerve trunk pain and deafferentiation pain. (For a description of classes, see Olesen, et al., The Headaches, pp. 9-14, Raven Press; see also, “Classification and diagnostic criteria for headache disorders, cranial neuralgias and facial Pain” Headache Classification Committee of the International Headache Society, Cephalalgia 8(supp. 7):1-96 (1988)).

### DETAILED DESCRIPTION OF THE INVENTION

I. Definitions

A. “Unit dose” or “unit dosage form” refers to a single drug administration entity. By way of example, a single tablet or capsule containing an extravasation inhibitor and an NSAID would be a unit dosage form.

B. “Migraine” refers to a well known medical condition characterized by recurrent severe headache that is often accompanied by other symptoms such as nausea, vomiting and heightened sensitivity to light or sound. Patients sometimes experience an “aura” that immediately precedes an attack during which they may experience alterations in vision, flashes of light or numbness. Migraine symptoms often subside upon treatment and then recur as a “relapse headache” within 48 hours.

C. “NK-1 antagonist” or “NK-1 receptor antagonist” refers to an agent that inhibits the biological activity induced by the binding of Substance P to the neurokinin-1 (NK-1) receptor. Substance P is a peptide 11 amino acids in length that is a member of the tachykinin family. It has been reported to participate in a wide variety of biological activities, including pain transmission.

D. “NSAIDs” refers to a group of nonsteroidal anti-inflammatory drugs that are well recognized in the art as analgesics and that act by inhibiting the activity of cyclooxygenase. In this way, the NSAIDs prevent the generation of pro-inflammatory prostaglandins. Acetaminophen also inhibits prostaglandin synthesis, but exhibits weak activity against the cyclooxygenase enzymes. Although the art does not generally recognize acetaminophen as an NSAID, unless otherwise indicated, it will be considered as an NSAID for the purposes of the present invention.

E. “Therapeutically effective” refers to a dosage of a drug or combination of drugs that provides the specific pharmacological response for which the drug or drugs have been administered in a significant number of subjects in need of such treatment. Thus, a therapeutically effective amount of an anti-inflammatory drug is a dosage sufficient to reduce the swelling or pain associated with inflammation. Similarly, a therapeutically effective dose of a drug administered to treat migraine would be an amount sufficient to reduce the pain or other symptoms that are associated with migraine.

F. “Onset of action” refers to the interval that begins when a drug is first ingested by a patient and that ends when a therapeutic effect is first observed.

G. “Therapeutic synergy” for a combination of an anti-inflammatory compound that acts by inhibiting protein extravasation (e.g., an NK-1 antagonist) and an NSAID means that, as measured in a population of patients, the combination exhibits one or more of the following: a) a longer duration of pain relief than that achievable using either drug alone, i.e., as the sole active agent; b) a greater reduction in pain severity than is achievable by the administration of either drug alone; and c) a reduction in one or more undesirable side effects associated with the administration of either drug alone. Among the side effects that may be reduced are: dizziness, gastrointestinal lesions, blood clots, stroke, heart attacks, ulcers, heart palpitations and discomfort caused by the constriction of blood vessels. When referring to migraine, the term “migraine-related therapeutic synergy” is used. This has the same definition as “therapeutic synergy,” except that the term “pain” refers to headache pain and an additional characteristic that may be indicative of synergy is: d) a reduction in the frequency of relapse headache that is greater than that achievable using either drug by alone.

H. “Anti-inflammatory compounds that act by blocking protein extravasation” are compounds showing activity in animal models of inflammation that measure the leakage of protein from blood vessels. In this regard, it should be noted that vasodilation and increased vascular permeability are two of the main characteristics that are associated with the inflammatory response. One inflammation assay that is accepted in the art and that measures protein extravasation has been described by Markowitz, et al. (J. Neurosci. 7:4129-4136 (1987)). This assay has been used both for 5-HT receptor agonists and for NK-1 antagonists (see May, et al., Curr. Opin. Neurol. 14:341-346 (2001)). As used herein, the term is intended to exclude NSAIDs.

I. “Co-timely” with respect to drug administration refers to the administration of a second drug for the treatment of a condition while a first drug is still present in a therapeutically effective amount.

J. “Coordinated” or “coordinated drug release” as used herein refers to the orderly, sequential release of drug agents from a dosage form. When referring to a composition containing gastric prokinetic agent, an NSAID and an NK-1 antagonist, a coordinated dosage form would release the gastric prokinetic agent first and the other drugs afterwards. More specifically, at least 80% of the total gastric prokinetic agent present in the dosage form should be released into the gastrointestinal tract at a time when less than 20% of NSAID or other anti-inflammatory compound has been released. The term can also be applied to a drug composition in which an NSAID and an anti-inflammatory compound that acts by blocking protein extravasation (e.g., an NK-1 antagonist) are present without metoclopramide. In this case, at least 80% of the NSAID should be released at a time when less than 20% of the anti-inflammatory compound is released or vice versa.

K. “Multilayer tablet” refers to a tablet dosage form in which components are found in two or more distinct regions. For example, a multilayer tablet may contain an outer layer in which NSAID is essentially the only active agent and an inner layer in which essentially the only active agent is an NK-1 antagonist.

II. Therapeutic Agents

A. NSAIDs

NSAIDs compatible with the present invention are well known in the art and are either commercially available or can be synthesized using standard techniques of medicinal chemistry. Although the dosage of NSAID may be adjusted by a clinician on a case-by-case basis, general guidelines have been established in the art for many of these compounds.

Examples of NSAIDs (with typical daily doses in parentheses) are as follows: propionic acids (fenoprofen (1500 mg); flurbiprofen (200 mg); suprofen; benoxaprofen; ibuprofen (1600 mg); ketoprofen (200 mg); naproxen (750 mg); and oxaprozin (1200 mg)); acetic acids (diclofenac (100 mg); aceclofenac (200 mg); etodolac (1200 mg); indomethacin (75-150 mg); ketorolac (10-30 mg)); ketones (nabumetone (1500 mg); sulindac (300 mg); tolmetin (800 mg)); fenamates (meclofenamate (400 mg); tolfenamic acid (400 mg); mefanamic acid); oxicams (droxicam; piroxicam (20 mg); lomoxicam (30 mg); meloxicam (15 mg); tenoxicam); salicylates (aspirin; diflunisal); pyrazolinates (oxyphenbutazone; azapropazone; phenylbutazone); COX-2 inhibitors (rofecoxib (50 mg); valdecoxib (20-40 mg); etorocoxib (60-120 mg); celecoxib (200 mg); lumiracoxib (100-200 mg); JTE-522; NS-398; and CS-502).

While the experienced clinician is able to monitor and adjust dosages for each patient relative to the severity of pain and the presence of side effects, approximate maximum daily dosages are as follows: flurbiprofen 300 mg; naproxen 1500 mg; naproxen sodium 1650 mg; oxaprozin 1800 mg; etodolac 1200 mg; indomethacin 150-200 mg; ketorolac 120 mg i.m. and 40 mg when taken orally; nabumetone 2000 mg; mefenamic acid 1000 mg; and piroxicam 20 mg. In particular instances, however, exceeding these “maximum” dosages may be the therapeutic choice of a medical professional.

B. NK-1 Receptor Antagonists

Examples of NK-1 antagonists that can be used in the present invention include: CP122721, CP96345 and CP96344 owned by Pfizer; R673 owned by Roche; SR140333, GR73632, GW679769, GW823296, and GW597599 (VESTIPITANT) owned by GlaxoSmithKline; TAK637 owned by Takeda; NKP608 owned by Novartis; RP67580 owned by Rhone Poulenc; and aprepitant owned by Merck.

One group of suitable NK-1 antagonists is described in U.S. Pat. No. 6,787,539 as having the structure of formula I: 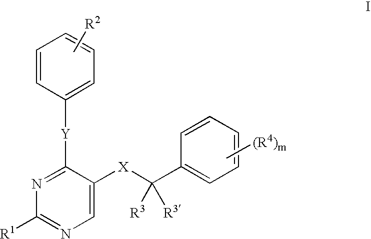
wherein
R¹ is lower alkyl, lower alkoxy, pyridinyl, pyrimidinyl, phenyl, —S-lower alkyl, —S(O)₂₋ lower alkyl, —N(R)—(CH₂)ₙ—N(R)₂, —O—(CH₂)ₙ—N(R)₂, —N(R)₂, or a cyclic tertiary amine of the group: 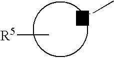
which may contain one additional heteroatom, selected from N, O or S, and wherein this group may be connected with the pyrimidine ring via the linker —O(CH₂)ₙ—;
R² is hydrogen, lower alkyl, lower alkoxy, halogen or trifluoromethyl;
R³/R³′ is, independently from each other, hydrogen or lower alkyl;
R⁴ is independently from each other halogen, trifluoromethyl or lower alkoxy;
R⁵ is hydrogen or lower alkyl;
R is, independently from each other, hydrogen or lower alkyl;
X is —C(O)N(R)— or —N(R)C(O);
Y is —O—, —S—, —SO₂—, or —N(R)—;
n is 1, 2, 3 or 4; and
m is 0, 1 or 2;
or a pharmaceutically acceptable acid addition salt thereof.

Specific examples of antagonists include:
2-(4-methyl-piperazin-1-yl)-4-o-tolyloxy-pyrimidine-5-carboxylic acid (3,5-bis-trifluoro methyl-benzyl)-methyl-amide;
2-piperazin-1-yl-4-o-tolyloxy-pyrimidine-5-carboxylic acid (3,5-bis-trifluoromethyl-benzyl)-methyl-amide;
4-(2-chloro-phenoxy)-2-(4-methyl-piperazin-1-yl)-pyrimidine-5-carboxylic acid (3,5-bis-tri fluoromethyl-benzyl)-methyl-amide;
2-(2-morpholin-4-yl-ethoxy)-4-o-tolyloxy-pyrimidine-5-carboxylic acid (3,5-bis-trifluoro methyl-benzyl)-methyl-amide;
2-(2-dimethylamino-ethoxy)-4-o-tolyloxy-pyrimidine-5-carboxylic acid (3,5-bis-trifluoro methyl-benzyl)-methyl-amide;
2-(3,5-bis-trifluoromethyl-phenyl)-N-methyl-N-(2-methylsulfanyl-4-o-tolyloxy-pyrimidin-5-yl)-isobutyramide;
2-(3,5-bis-trifluoromethyl-phenyl)-N-[4-(4-fluoro-phenoxy)-2-methylsulfanyl-pyrimidin-5-yl]-N-methyl-isobutyramide;
2-(3,5-bis-trifluoromethyl-phenyl)-N-[4-(2-chloro-phenoxy)-2-methylsulfanyl-pyrimidin-5-yl]-N-methyl-isobutyramide;
2-(3,5-bis-trifluoromethyl-phenyl)-N-methyl-N-(2-morpholin-4-yl-4-o-tolyloxy-pyrimidin-5-yl)-isobutyramide;
2-(3,5-bis-trifluoromethyl-phenyl)-N-methyl-N-[2-(4-methyl-piperazin-1-yl)-4-o-tolyloxy-pyrimidin-5-yl]-isobutyramide;
2-(3,5-bis-trifluoromethyl-phenyl)-N-methyl-N-(2-piperazin-1-yl-4-o-tolyloxy-pyrimidin-5-yl)-isobutyramide;
2-(3,5-bis-trifluoromethyl-phenyl)-N-[4-(4-fluoro-phenoxy)-2-(4-methyl-piperazin-1-yl)-pyrimidin-5-yl]-N-methyl-isobutyramide;
2-(3,5-bis-trifluoromethyl-phenyl)-N-[4-(4-fluoro-phenoxy)-2-piperazin-1-yl-pyrimidin-5-yl]-N-methyl-isobutyramide;
2-(3,5-bis-trifluoromethyl-phenyl)-N-[4-(2-chloro-phenoxy)-2-morpholin-4-yl-pyrimidin-5-yl]-N-methyl-isobutyramide;
2-(3,5-bis-trifluoromethyl-phenyl)-N-[4-(2-chloro-phenoxy)-2-(4-methyl-piperazin-1-yl)-pyrimidin-5-yl]-N-methyl-isobutyramide;
2-(3,5-Bis-trifluoromethyl-phenyl)-N-[4-(2-chloro-phenoxy)-2-piperazin-1-yl-pyrimidin-5-yl]-N-methyl-isobutyramide;
2-(3,5-bis-trifluoromethyl-phenyl)-N-[2-(2-dimethylamino-ethylamino)-4-o-tolyloxy-pyrimidin-5-yl]-N-methyl-isobutyramide;
2-(3,5-bis-trifluoromethyl-phenyl)-N-[2-(2-dimethylamino-ethylamino)-4-(4-fluoro-phenoxy)-pyrimidin-5-yl]-N-methyl-isobutyramide;
2-(3,5-bis-trifluoromethyl-phenyl)-N-methyl-N-[2-(2-morpholin-4-yl-ethoxy)-4-o-tolyl oxy-pyrimidin-5-yl]-isobutyramide;
2-(3,5-bis-trifluoromethyl-phenyl)-N-[4-(2-chloro-phenoxy)-2-(2-morpholin-4-yl-ethoxy)-pyrimidin-5-yl]-N-methyl-isobutyramide;
2-(3,5-bis-trifluoromethyl-phenyl)-N-[2-(2-dimethylamino-ethoxy)-4-o-tolyloxy-pyrimidin-5-yl]-N-methyl-isobutyramide;
2-(3,5-bis-trifluoromethyl-phenyl)-N-[2-(3-dimethylamino-propoxy)-4-o-tolyloxy-pyrimidin-5-yl]-N-methyl-isobutyramide;
2-(3,5-bis-trifluoromethyl-phenyl)-N-[2-(2-dimethylamino-ethoxy)-4-(4-fluoro-phenoxy)-pyrimidin-5-yl]-N-methyl-isobutyramide;
2-(3,5-bis-trifluoromethyl-phenyl)-N-[2-(3-dimethylamino-propoxy)-4-(4-fluoro-phenoxy)-pyrimidin-5-yl]-N-methyl-isobutyramide;
2-(3,5-bis-trifluoromethyl-phenyl)-N-[2-(2-dimethylamino-ethoxy)-4-(2-chloro-phenoxy)-pyrimidin-5-yl]-N-methyl-isobutyramide; and
2-(3,5-bis-trifluoromethyl-phenyl)-N-[2-(3-dimethylamino-propoxy)-4-(2-chloro-phenoxy)-pyrimidin-5-yl]-N-methyl-isobutyramide.

U.S. Pat. No. 6,727,249 describes azabicyclic ether derivatives that can serve as NK-1 antagonists. Similarly, U.S. Pat. No. 6,642,226 describes substituted phenyl-piperidine methanone compounds such as:
rac-cis-(3,5-bis-trifluoromethyl-phenyl)-[4-(4-morpholin-4-yl-phenyl)-3-phenyl-piperidin-1-yl]-methanone;
rac-cis-(3,5-bis-trifluoromethyl-phenyl)-{4-[4-(4-methyl-piperazin-1-yl)-phenyl]-3-phenyl-piperidin-1-yl}-methanone;
rac-cis-(3,5-bis-trifluoromethyl-phenyl)-[3-(4-chloro-phenyl)-4-phenyl-piperidin-1-yl]-methanone;
rac-cis-(3,5-bis-trifluoromethyl-phenyl)-[4-(3-bromo-phenyl)-3-phenyl-piperidin-1-yl]-methanone;
rac-cis-(3,5-bis-trifluoromethyl-phenyl)-{4-[4-(2-methoxy-ethylamino)-phenyl]-3-phenyl-piperidin-1-yl}-methanone;
rac-cis-(3,5-bis-trifluoromethyl-phenyl)-[3-(4-chloro-phenyl)-4-morpholin-4-yl-piperidin-1-yl]-methanone;
rac-cis-(3,5-bis-trifluoromethyl-phenyl)-(4-morpholin-4-yl-3-phenyl-piperidin-1-yl)-methanone;
rac-cis-(3,5-bis-trifluoromethyl-phenyl)-[3-(4-fluoro-phenyl)-4-morpholin-4-yl-piperidin-1-yl]-methanone;
rac-cis-(3,5-bis-trifluoromethyl-phenyl)-[3′-(4-chloro-phenyl)-4-morpholin-4-yl-[1,4′]bi piperidinyl-1′-yl]-methanone;
rac-cis-(3,5-bis-trifluoromethyl-phenyl)-[4-(4-methyl-piperazin-1-yl)-3-phenyl-piperidin-1-yl]-methanone;
rac-cis-(3,5-bis-trifluoromethyl-phenyl)-(3-phenyl-4-piperazin-1-yl-piperidin-1-yl)-methanone;
rac-cis-2 {4-[1-(3,5-bis-trifluoromethyl-benzoyl)-3-phenyl-piperidin-4-yl]-piperazin-1-yl}-N,N-dimethyl-acetamide;
rac-cis-(3,5-bis-trifluoromethyl-phenyl)-[4-(4-cyclopropylmethyl-piperazin-1-yl)-3-phenyl-piperidin-1-yl]-methanone;
rac-cis-[4-(4-benzyl-piperazin-1-yl)-3-phenyl-piperidin-1-yl]-(3,5-bis-trifluoromethyl-phenyl)-methanone;
rac-cis-(3,5-bis-trifluoromethyl-phenyl)-[4-(4-cyclopropanecarbonyl-piperazin-1-yl)-3-phenyl-piperidin-1-yl]-methanone;
rac-cis-{4-[1-(3,5-bis-trifluoromethyl-benzoyl)-3-phenyl-piperidin-4-yl]-piperazin-1-yl}-morpholin-4-yl-methanone;
rac-cis-(3,5-bis-trifluoromethyl-phenyl)-[4-(4-cyclopropyl-piperazin-1-yl)-3-phenyl-piperidin-1-yl]-methanone;
rac-cis-(3,5-bis-trifluoromethyl-phenyl)-[3-(4-fluoro-phenyl)-4-(4-methyl-piperazin-1-yl)-piperidin-1-yl]-methanone;
rac-cis-2-{4-[1-(3,5-bis-trifluoromethyl-benzoyl)-3-phenyl-piperidin-4-yl]-piperazin-1-yl}-N-(2,6-dimethyl-phenyl)-acetamide;
rac-cis-(3,5-bis-trifluoromethyl-phenyl)-[3-phenyl-4-(2,3,5,6-tetrahydro-[1,2′]bipyrazinyl-4-yl)-piperidin-1-yl]-methanone;
(+)-(3,5-bis-trifluoromethyl-phenyl)-[4-(4-cyclopropanecarbonyl-piperazin-1-yl)-3-phenyl-piperidin-1-yl]-methanone;
rac-cis-2-{4-[1-(3,5-bis-trifluoromethyl-benzoyl)-3-phenyl-piperidin-4-yl]-piperazin-1-yl}-N-(4-fluoro-phenyl)-acetamide;
rac-cis-1-{4-[1-(3,5-bis-trifluoromethyl-benzoyl)-3-phenyl-piperidin-4-yl]-piperazin-1-yl}-2-phenyl-ethanone;
rac-cis-(3,5-bis-trifluoromethyl-phenyl)-[3-(4-fluoro-phenyl)-4-piperazin-1-yl-piperidin-1-yl]-methanone;
rac-cis-(3,5-bis-trifluoromethyl-phenyl)-[4-(4-cyclopropylmethyl-piperazin-1-yl)-3-(4-fluoro-phenyl)-piperidin-1-yl]-methanone;
(−)-(3,5-bis-trifluoromethyl-phenyl)-[3-(4-fluoro-phenyl)-4-(4-methyl-piperazin-1-yl)-piperidin-1-yl]-methanone;
(−)-(3,5-bis-trifluoromethyl-phenyl)-[3-phenyl-4-(4-methyl-piperazin-1-yl)-piperidin-1-yl]-methanone;
(−)-4-(3,5-bis-trifluoromethyl-phenyl)-[4-(4-cyclopropylmethyl-piperazin-1-yl)-3-p-tolyl-piperidin-1-yl]-methanone;
(−)-(3,5-bis-trifluoromethyl-phenyl)-[4-(4-cyclopropanecarbonyl-piperazin 1-yl)-3-p-tolyl-piperidin-1-yl]-methanone;
(−)-(3,5-bis-trifluoromethyl-phenyl)-{4-[4-(morpholine-4-carbonyl)-piperazin-1-yl]-3-p-tolyl-piperidin-1-yl}-methanone;
rac-cis-1-{4-[1-(3,5-bis-trifluoromethyl-benzoyl)-3-(4-chloro-phenyl)-piperidin-4-yl]-piperazin-1-yl}-2,2,2-trifluoro-ethanone;
(−)-1-{4-[1-(3,5-bis-trifluoromethyl-benzoyl)-3-(4-chloro-phenyl)-piperidin-4-yl]-piperazin-1-yl}-2,2,2-trifluoro-ethanone;
(−)-(3,5-bis-trifluoromethyl-phenyl)-[4-(4-cyclopropyl-piperazin-1-yl)-3-p-tolyl-piperidin-1-yl]-methanone;
(−)-(3,5-bis-trifluoromethyl-phenyl)-[4-(4-methyl-piperazin-1-yl)-3-p-tolyl-piperidin-1-yl]-methanone;
(−)-(3,5-bis-trifluoromethyl-phenyl)-[3-(4-chloro-phenyl)-4-(4-cyclopropylmethyl-piperazin-1-yl)-piperidin-1-yl]-methanone;
(−)-(3,5-bis-trifluoromethyl-phenyl)-[4-(4-cyclopropylmethyl-piperazin-1-yl)-3-phenyl-piperidin-1-yl]-methanone;
(−)-(3,5-bis-trifluoromethyl-phenyl)-{4-[4-(2-hydroxy-ethyl)-piperazin-1-yl]-3-phenyl-piperidin-1-yl}-methanone;
(−)-(3,5-bis-trifluoromethyl-phenyl)-[4-(4-cyclopropyl-piperazin-1-yl)-3-phenyl-piperidin-1-yl]-methanone;
rac-cis-(3,5-bis-trifluoromethyl-phenyl)-(4,4-difluoro-3′-phenyl-[1,4′]bipiperidinyl-1′-yl)-methanone;
rac-cis-(3,5-bis-trifluoromethyl-phenyl)-[3′-(4-fluoro-phenyl)-3-hydroxy-[1,4′]bipiperidinyl-1′-yl]-methanone;
rac-cis-(3,5-bis-trifluoromethyl-phenyl)-(4-hydroxy-3′-phenyl-[1,4′]bipiperidinyl-1′-yl)-methanone;
rac-cis-(3,5-bis-trifluoromethyl-phenyl)-(4-cyclopropylmethoxy-3′-phenyl-[1,4′]bipiperidinyl-1′-yl)-methanone;
rac-cis-1′-(3,5-bis-trifluoromethyl-benzoyl)-3′-phenyl-[1,4′]bipiperidinyl-4-one;
rac-cis-(3,5-bis-trifluoromethyl-phenyl)-[3′-(4-chloro-phenyl)-4-hydroxy-[1,4′]bipiperidinyl-1′-yl]-methanone;
rac-cis-(3,5-bis-trifluoromethyl-phenyl)-[3′-(4-chloro-phenyl)-4-cyclopropylmethoxy-[
1,4′]bipiperidinyl-1′-yl]-methanone;
(3RS,3′RS,4SR)- and (3RS,3′SR,4SR)-cyclopropanecarboxylic acid [1′-(3,5-bis-trifluoromethyl-benzoyl)-3′-(4-fluoro-phenyl)-[1,4′]bipiperidinyl-3-yl]-amide;
(3RS,3′RS,4SR)- and (3RS,3′SR,4SR)-N-[1′-(3,5-bis-trifluoromethyl-benzoyl)-3′-(4-fluoro-phenyl)-[1,4′]bipiperidinyl-3-yl]-2-phenyl-acetamide;
rac-cis-(3,5-bis-trifluoromethyl-phenyl)-[3′-(4-chloro-phenyl)-4-dimethylamino-[1,4′]bipiperidinyl-1′-yl]-methanone;
rac-cis-(3,5-bis-trifluoromethyl-phenyl)-[3′-(4-chloro-phenyl)-4-cyclopropylamino-[1,4′]bipiperidinyl-1′-yl]-methanone;
(3R,3′R,4R)- and (3S,3′R,4S)-(3,5-bis-trifluoromethyl-phenyl)-[4-(3′-hydroxy-pyrrolidin-1′-yl)-3-phenyl-piperidin-1-yl]-methanone;
(3R,3′R,4R)- and (3S,3′R¹⁴S)-(3,5-bis-trifluoromethyl-phenyl)-[4-(3-cyclopropylmethoxy-pyrrolidin-1-yl)-3-phenyl-piperidin-1-yl]-methanone;
rac-cis-1-[1-(3,5-bis-trifluoromethyl-benzoyl)-3-phenyl-piperidin-4-yl]-pyrrolidin-3-one;
(−)-(3,5-bis-trifluoromethyl-phenyl)-[3-(4-chloro-phenyl)-4-pyrrolidin-1-yl-piperidin-1-yl]-methanone;
(3RS,3′RS,4SR)— and (3RS,3′SR,4SR)-[4-(3-amino-pyrrolidin-1-yl)-3-(4-fluoro-phenyl)-piperidin-1-yl]-(3,5-bis-trifluoromethyl-phenyl)-methanone;
rac-cis-(3,5-bis-trifluoromethyl-phenyl)-[4-(1-oxo-114-thiomorpholin-4-yl)-3-phenyl-piperidin-1-yl]-methanone; and
rac-cis-(3,5-bis-trifluoromethyl-phenyl)-[4-(1,1-dioxo-116-thiomorpholin-4-yl)-3-phenyl-piperidin-1-yl]-methanone.
U.S. Pat. No. 6,770,637 describes substituted 4-phenyl-pyridine compounds such as:
N-(3,5-bis-trifluoromethyl-benzyl)-6-(4-hydroxyacetyl-piperazin-1-yl)-N-met hyl-4-o-tolyl-nicotinamide;
N-(3,5-bis-trifluoromethyl-benzyl)-6-chloro-N-methyl-4-o-tolyl-nicotinamide;
N-(3,5-bis-trifluoromethyl-benzyl)-6-cyanomethyl-N-methyl-4-o-tolyl-nicotinamide;
(RS)—N-(3,5-bis-trifluoromethyl-benzyl)-6-(2-hydroxy-ethanesulfinylmethyl)-N-methyl-4-o-tolyl-nicotinamide;
N-(3,5-bis-trifluoromethyl-benzyl)-N-methyl-6-(1-methyl-1H-imidazol-2-ylsulfanylmethyl)-4-o-tolyl-nicotinamide;
(RS)-N-(3,5-bis-trifluoromethyl-benzyl)-N-methyl-6-(pyridine-2-sulfinyl)-4-o-tolyl-nicotinamide;
N-(3,5-bis-trifluoromethyl-benzyl)-N-methyl-6-(pyridine-2-sulfonyl)-4-o-tolyl-nicotinamide; and
N-(3,5-bis-trifluoromethyl-benzyl)-6-(3-hydroxy-propoxy)-N-methyl-4-o-tolyl-nicotinamide.

Other US patent references describing NK-1 antagonists suitable for use in the invention include: U.S. Pat. No. 6,756,380; U.S. Pat. No. 6,747,026; U.S. Pat. No. 6,376,507; U.S. Pat. No. 6,194,436; U.S. Pat. No. 6,194,436; U.S. Pat. No. 6,162,339; U.S. Pat. No. 6,576,762; U.S. Pat. No. 6,552,088; U.S. Pat. No. 6,458,830; U.S. Pat. No. 6,489,343; U.S. Pat. No. 6,452,001; U.S. Pat. No. 6,407,111; U.S. Pat. No. 6,274,588; U.S. Pat. No. 6,225,316; U.S. Pat. No. 6,596,773; U.S. Pat. No. 6,599,900; U.S. Pat. No. 6,482,829; U.S. Pat. No. 6,576,762; U.S. Pat. No. 6,593,472; U.S. Pat. No. 6,136,824; U.S. Pat. No. 5,716,965; U.S. Pat. No. 5,852,038; U.S. Pat. No. 5,807,867; U.S. Pat. No. 5,886,009; U.S. Pat. No. 5,939,433; U.S. Pat. No. 5,773,450; U.S. Pat. No. 5,744,480; U.S. Pat. No. 5,232,929; U.S. Pat. No. 5,332,817; U.S. Pat. No. 5,122,525; U.S. Pat. No. 5,843,966; U.S. Pat. No. 5,703,240; U.S. Pat. No. 5,719,147; U.S. Pat. No. 5,637,699; and U.S. Pat. No. 5,610,176;

Suitable NK-1 receptor antagonists are also described in published European Patent references: EP 0 360 390; EP 0 394 989; EP 0 429 366; EP 0 443 132; EP 0 482 539: EP 0 512901; EP0512902; EP0514273; EP0514275; EP0517589; EP0520555; EP0 522808; EP0528495; EP0532456; EP0533280; EP0536817; EP0545478EP0 577394; EP0590 152; EP0599538; EP0610793; EP0634402; EP0686629; EP0 693 489; EP 0 694 535; EP 0 699 655; EP 0 699 674; EP 0 707 006; EP 0 708 101; EP 0 714 891; EP 0 723 959; EP 0 733 632; and EP 0 776 893.

International applications describing NK-1 antagonists include 90/05525, 91/09844, 91/18899, 92/01688, 92/06079, 92/12151, 92/15585, 92/17449, 92/20661, 92/20676, 92/21677, 93/00330, 93/00331, 93/01159, 93/01165, 93/01169, 93/01170, 93/06099, 93/09116, 93/10073, 93/14113, 93/18023, 93/19064, 93/21155, 9321181, 93/23380, 93/24465, 94/01402, 94/02461, 94/03429, 94/03445, 94/04494, 94/04496, 94/05625, 94/07843, 94/10165, 94/10167, 94/10168, 94/10170, 94/11368, 94/13639, 94/13663, 94/14767, 94/15903, 94/19320, 94/19323, 94/20500, 94/26735, 94/26740, 94/29309, 95/02595, 95/04040, 95/04042, 95/06645, 95/07886, 95/07908, 95/08549, 95/11880, 95/14017, 95/15311, 95/16679, 95/17382, 95/18124, 95/18129, 95/19344, 95/20575, 95/21819, 96/22525, 95/23798, 95/26338, 95/28418, 95/30674, 95/30687, 96/05193, 96/05203, 96/06094, 96/07649, 96/10562, 96/16939, 96/18643, 96/20197, 96/21661, 96/29304, 96/29317, 96/29326, 96/29328, 96/31214, 96/32385, 96/37489, 97/01553, 97/01554, 97/03066, 97/08144, 97/14671, 97/17362, 97/18206, 97/19084, 97/19942, 90/05729, and 97/21702.

III. Making of Pharmaceutical Compositions

Pharmaceutical compositions and dosage forms can be produced in accordance with conventional methods that are standard in the art (see, e.g., Remington's Pharmaceutical Sciences, 16^{th} ed., A. Oslo. editor, Easton, Pa. (1980)). Active ingredients, individually or in combination, may be mixed with pharmaceutically acceptable organic or inorganic carrier substances suitable for parenteral, enteral (e.g., oral or intranasal) or topical application. Pharmaceutically acceptable carriers include, but are not limited to: water; salt solutions; alcohols; gum arabic; vegetable oils; benzyl alcohols; polyethylene glycols; gelatin; carbohydrates, such as lactose, amylase, or starch; magnesium stearate; talc; titanium dioxide; silicic acid; viscous paraffin; perfume oil; fatty acid esters; etc.

The pharmaceutical preparations can be sterilized and, if desired, mixed with auxiliary agents, e.g., lubricants, preservatives, stabilizers, wetting agents, emulsifiers, salts for controlling osmotic pressure, buffers, coloring agents, flavoring agents, and/or aromatic substances. Compositions can also include other active agents.

If desired, enteric coating layers may be incorporated into tablets or capsules. Coating materials may include one or more of the following: methacrylic acid copolymers, shellac, hydroxypropylmethylcellulose phthalate, polyvinyl acetate phthalate, hydroxy-propylmethylcellulose trimellitate, carboxymethylethylcellulose, cellulose acetate phthalate, or other suitable enteric coating polymers. The pH at which the enteric coat will dissolve can be controlled by the polymer or combination of polymers selected and/or ratio of pendant groups. For example, dissolution characteristics may be affected by the ratio of free carboxyl groups to ester groups. Enteric coating layers may also contain pharmaceutically acceptable plasticizers such as triethyl citrate, dibutyl phthalate, tricetin, polyethylene glycols, polysorbates, etc. Additives, such as dispersants, colorants, anti-adhering and anti-foaming agents, may also be included.

IV. Making of Tablet Dosage Forms

Although the present invention is compatible with any route of administration, oral dosage forms are preferred, with the most preferred being tablets or capsules. Methods for making multilayer tablets are well known in the art and procedures for making coordinated dosage forms, particularly with compositions containing metoclopramide, have been previously described (see U.S. Pat. No. 6,479,551). In a multilayer configuration, one portion of a tablet or capsule will contain an NSAID, anti-inflammatory agent, or gastric prokinetic agent. Other portions will contain the other drugs along with appropriate excipients, dissolution agents, lubricants, fillers, etc. Tablets may be granulated by methods such as slugging, low- or high-sheer granulation, wet granulation, or fluidized-bed granulation. Of these processes, slugging generally produces tablets of less hardness and greater friability. Low-sheer granulation, high-sheer granulation, wet granulation and fluidized bed granulation generally produce harder, less friable tablets.

All references cited herein are fully incorporated by reference. Having now fully described the invention, it will be understood by those of skill in the art that the invention may be practiced within a wide and equivalent range of conditions, parameters and the like, without affecting the spirit or scope of the invention or any embodiment thereof.

What is claimed is:

1. A pharmaceutical composition in unit dose form, comprising:
a) an anti-inflammatory compound that acts by blocking protein extravasation; and
b) an NSAID;
wherein said anti-inflammatory compound and said NSAID are present in an amount effective to relieve pain upon the administration of one or more unit doses to a patient.

2. The pharmaceutical composition of claim 1, wherein said anti-inflammatory compound of paragraph a) is a NK-1 receptor antagonist.

3. The pharmaceutical composition of claim 2, wherein said NSAID has an onset of action of two hours or less.

4. The pharmaceutical composition of claim 2, wherein said anti-inflammatory compound and said NSAID are present in an amount sufficient to provide therapeutic synergy or migraine-related therapeutic synergy and wherein no more than one drug is present in an amount sufficient to provide a therapeutic effect in the absence of the other.

5. The pharmaceutical composition of claim 2, wherein said anti-inflammatory compound of paragraph a) is present in said unit dosage form at between 0.5 and 600 mg.

6. The pharmaceutical composition of claim 5, wherein said NSAID is present in said unit dosage form at 1-600 mg.

7. The pharmaceutical composition of claim 6, wherein said NSAID is selected from the group consisting of: aspirin; ibuprofen; acetaminophen; naproxen; diclofenac; ketorolac; etodolac; flurbiprofen; oxaprozin; indomethacin; mefenamic acid; nabumetone; piroxicam; celecoxib; rofecoxib; valdecoxib; and lornoxicam.

8. The pharmaceutical composition of claim 1, wherein:
a) said anti-inflammatory compound is an NK-1 antagonist at 1-600 mg; and
b) said NSAID is present at 1-600 mg, and is selected from the group consisting of: aspirin; ibuprofen; acetaminophen; naproxen; diclofenac; ketorolac; lomoxicam; and etodolac.

9. A method of treating a patient for pain, inflammation or migraine, comprising: administering to said patient a therapeutically effective amount of the pharmaceutical composition of claim 1.

10. The method of claim 9, wherein said anti-inflammatory compound in said pharmaceutical composition is an NK-1 receptor antagonist.

11. The method of claim 10, wherein said NSAID in said pharmaceutical composition is selected from the group consisting of: aspirin; ibuprofen; acetaminophen; naproxen; diclofenac; ketorolac; etodolac; flurbiprofen; oxaprozin; indomethacin; mefenamic acid; nabumetone; piroxicam; celecoxib; rofecoxib; valdecoxib; and lomoxicam.

12. A method of treating a patient for pain, inflammation or migraine, comprising:
a) administering to said patient an NSAID; and
b) administering to said patient an anti-inflammatory compound that acts by blocking protein extravasation,
wherein said NSAID of step a) and said anti-inflammatory compound of step b) are administered in a co-timely manner and are administered in a therapeutically effective amount.

13. The method of claim 12, wherein said anti-inflammatory compound of step b) is administered within two hours of the NSAID of step a).

14. The method of claim 13, wherein said anti-inflammatory compound of step b) is an NK-1 antagonist.

15. The method of claim 14, wherein said NK-1 antagonist is administered within two hours of said NSAID.

16. The method of claim 15, wherein said NK-1 antagonist is administered within one hour of said NSAID.

17. The method of claim 15, wherein said NSAID and said anti-inflammatory compound of are administered in an amount sufficient to provide for therapeutic synergy or migraine-related therapeutic synergy and wherein no more than one drug is administered in an amount sufficient to provide a therapeutic effect in the absence of the other.

18. The method of claim 17, wherein said NSAID and said anti-inflammatory compound are each administered at a dose of 1-600 mg.

19. The method of claim 18, wherein said NSAID is selected from the group consisting of: aspirin; ibuprofen; acetaminophen; naproxen; diclofenac; ketorolac; etodolac; flurbiprofen; oxaprozin; indomethacin; mefenamic acid; nabumetone; piroxicam; celecoxib; rofecoxib; valdecoxib; and lomoxicam.

20. The method of claim 19, wherein said patient is treated for migraine.
